(19) United States
(12) Patent Application Publication
(10) Pub. No.: US 2024/0216708 A1
(43) Pub. Date: Jul. 4, 2024

(54) Diagnostic and Therapeutic Array for Treatment of Skin Cancer

(71) Applicant: NFI, LLC, Madison, WI (US)

(72) Inventors: Daniel van der Weide, Madison, WI (US); Yuchen Gu, Madison, WI (US)

(73) Assignee: NFI, LLC, Madison, WI (US)

(21) Appl. No.: 18/531,584

(22) Filed: Dec. 6, 2023

Related U.S. Application Data
(60) Provisional application No. 63/430,655, filed on Dec. 6, 2022.

(51) Int. Cl. A61N 1/40; A61B 5/0531

(52) U.S. Cl. CPC A61N 1/40; A61B 5/0531

(57) ABSTRACT

In an illustrative embodiment, a dual mode, high-sensitivity, and low-cost applicator array based on split-ring resonators (SRR) and microstrip coupled lines serves as both an imaging and treatment tool for skin cancers. The applicator array consists of 3×3 unit cells, in which each unit in a row is tuned to a separate frequency, ranging from 8 to 15 GHz (unloaded). Via fences surrounding the unit cells provide E-field shielding and enhance resonance. The excitation is coupled magnetically from microstrip to SRRs and generates strong E-fields across the gap between SRR loop terminals. By observing the resonance shift and attenuation under different material under test (MUT), skin in one case, permittivity differences can be analyzed to distinguish malignancies from healthy tissue. Using the same applicator array, hyperthermia capability requires less than 5 W of power to cause significant temperature elevation.

### CROSS-REFERENCE TO RELATED APPLICATIONS

This application claims the benefit and priority of U.S. Prov. Pat. App. Ser. No. 63/430,655, titled Diagnostic and Therapeutic Array for Skin Cancer Treatment, filed Dec. 6, 2022, which is incorporated by reference herein.

### BACKGROUND

Skin cancer is increasingly common in the United States in part due to migration to sunnier climates and lack of proper protection. It is estimated that one-fifth of the population in the U.S. will develop skin cancer in their lifetime; nearly one million Americans are living with melanoma, one of the deadliest skin malignancies. Early detection and treatment of skin cancer is the best approach to improve awareness and prevent local invasion or metastasis.

In U.S. Pat. No. 7,725,151, incorporated herein by reference, one of the present inventors disclosed microwave/millimeter wave probes for detecting differences in characteristics between healthy and unhealthy tissues. Various researchers have demonstrated single open-ended waveguide/probes capable of detecting malignancies. Due to manufacturing costs and small contact area of such sensors, however, such probes are less economical and efficient for conducting thorough imaging on a large area of skin. Other researchers use probes similar to antennas for remote sensing. Since such probes require maintaining constant distance between the probe and tissue under test, they are less useful for clinical use.

Metamaterial structures, especially split-ring resonators (SRR), have attracted considerable attention in the past decade and enabled applications such as temperature and humidity monitoring. More recently, due to their highly focused field for sensing, they have also been used in permittivity extraction for dielectric and biological materials. In M. Baghelani, N. Hosseini, and M. Daneshmand, “Selective Measurement of Water Content in Multivariable Biofuel Using Microstrip Split Ring Resonators,” 2020 IEEE MTT-S International Microwave Symposium (IMS), vol. 00, pp. 225-228, 2020, a highly sensitive SRR is proposed, distinguishing among various liquids with different dielectric constants. In M. Puentes, M. Maasch, M. Schüβler, and R. Jakoby, “Frequency Multiplexed 2-Dimensional Sensor Array Based on Split-Ring Resonators for Organic Tissue Analysis,” IEEE Trans Microwave Theory Tech, vol. 60, no. 6, pp. 1720-1727, 2012 an SRR based sensing array is proposed, sensing and treating skin samples at 1 GHz. Due to its low operating frequency and large sensor size, it is not yet capable of sufficient resolution to detect and treat actual skin cancer lesions.

### SUMMARY OF ILLUSTRATIVE EMBODIMENTS

In one aspect, the present disclosure relates to systems and methods for an applicator array consisting of compact resonators, providing local sensing regions as small as a fraction of a millimeter while capable of treating local lesions with hyperthermia. In one embodiment the compact resonators comprise split-ring resonators (SRRs), fabricated using modern PCB technology on FR-4, and that operate between 8-15 GHz (unloaded). The proposed sensor can support early management of skin cancers for primary care physicians and entry-level practitioners, as both a non-invasive imaging modality and treatment tool due to its compact footprint, affordability, and ease of use.

The foregoing general description of the illustrative implementations and the following detailed description thereof are merely exemplary aspects of the teachings of this disclosure and are not restrictive.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are incorporated in and constitute a part of the specification, illustrate one or more embodiments and, together with the description, explain these embodiments. The accompanying drawings have not necessarily been drawn to scale. Any values dimensions illustrated in the accompanying graphs and figures are for illustration purposes only and may or may not represent actual or preferred values or dimensions. Where applicable, some or all features may not be illustrated to assist in the description of underlying features. In the drawings:

FIG. 1 shows a partial cross-section of a sensor circuit according to an embodiment;

FIG. 2 shows a top view of a sensor circuit according to an embodiment;

FIG. 3 graphs measured resonance for three difference board structures and four different loop shapes;

FIG. 4 graphs resonance frequency shifts vs. material-under-test permittivity for the four different loop shapes;

FIG. 5 illustrates an equivalent circuit for a line of three different SRRs coupled to the same microstrip transmission line;

FIG. 6 graphs measured attenuation vs. frequency for one SRR and a variety of different tissue samples;

FIG. 7 graphs measured attenuation vs. frequency for one SRR under a variety of contact force levels;

FIG. 8 depicts an embodiment of a hand-held applicator for a dual-mode medical device according to an embodiment; and

FIG. 9 contains a block diagram for a dual-mode medical device according to an embodiment.

### DETAILED DESCRIPTION OF ILLUSTRATIVE EMBODIMENTS

The description set forth below in connection with the appended drawings is intended to be a description of various, illustrative embodiments of the disclosed subject matter. Specific features and functionalities are described in connection with each illustrative embodiment; however, it will be apparent to those skilled in the art that the disclosed embodiments may be practiced without each of those specific features and functionalities.

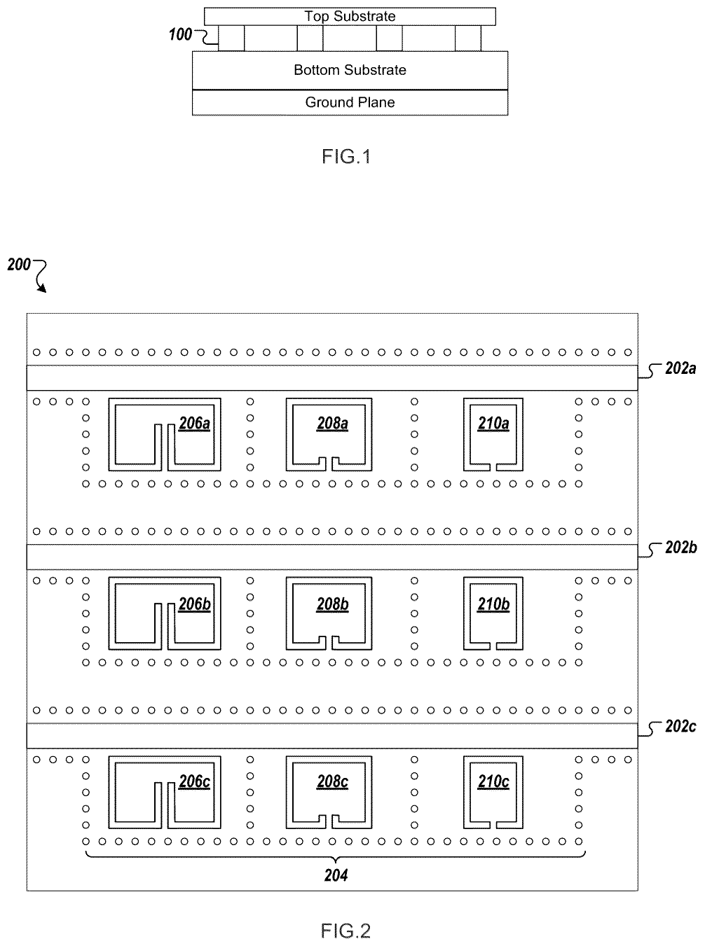

The applicator array board is fabricated on a two-layer Printed Circuit Board (PCB) with nine unit cells divided into three parallel rows with independent microstrip lines, shown in FIGS. 1 and 2. As shown in the cross-section of FIG. 1, a bottom substrate is plated with a ground plane on its underside, and the SRR array and microstrip transmission lines are patterned on the top side of the bottom substrate. An additional top dielectric layer of substrate material is added to the top of the SRRs (e.g., 100) for isolation from materials under test (MUT).

FIG. 2 shows a plan view of a sensor array 200, illustrating the structure visible on the top side of the bottom substrate. Three microstrip transmission lines 202a, 202b, 202c are spaced from top to bottom on array 200. Each respective transmission line couples to three SRRs (e.g., 202a couples to SRRs 206a, 208a, and 210a), with each SRR having a different configuration from its neighbors along a same transmission line.

Since each SRR emits E-field across its gap within a few millimeters of each other, mutual coupling is another concern for the array structure. To address the potential E-field leakage, via fences around the SRRs and transmission lines further improve E-field shielding for performance, as shown in FIG. 2. The via fences consist of lines of through vias 204 that connect through the bottom substrate to the ground plane.

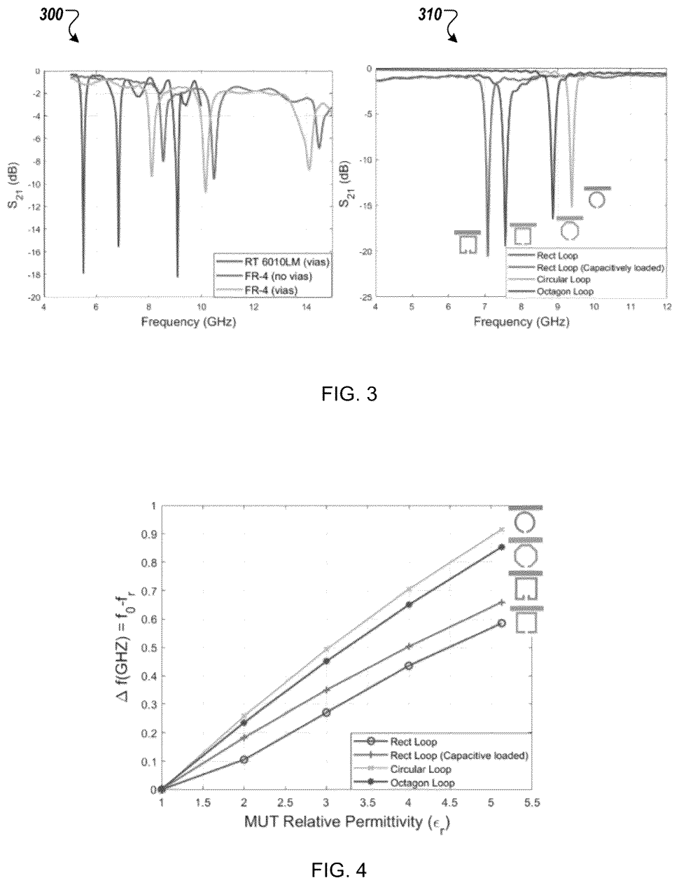

To achieve desired resonance and quality factor at each unit cell, the unloaded sensor was simulated on each of two different substrates. The result is shown in FIG. 3 graph 300. Though RT 6010LM (εr=10.2) substrate offers higher Q and greater field concentration due to its higher dielectric constant, the substrate using FR-4 (εr=4.6) also provides sufficient performance. Graph 300 also shows the difference in response for FR-4 with and without the via fences.

Different sensor geometries offer different resonance and sensitivity characteristics. Four different SRRs are simulated on RT 6010LM using the same design variables including footprint size (length and width), metal thickness/width, sensing gap width and distance to microstrip line. The simulation results in FIG. 3 graph 310 demonstrate that rectangular loops with capacitively loaded bars and circular loops are presently preferred for the best resonance and sensitivity, although other shapes are potential alternatives. To prevent excessive frequency shifting of each SRR under loaded conditions resulting in possible overlap with resonances of other units, rectangular loops with bars (capacitively loaded) were implemented for a balance of resonance and sensitivity. The graph of FIG. 4 compares the loaded frequency shifts for each of the four loop shapes, as a function of the dielectric constant of the material under test (MUT).

Since the demonstrated effects are primarily geometry dependent (not highly material dependent), considering cost, performance and availability of materials, an acceptable embodiment can be implemented on FR-4 substrate. Detailed dimensions for this embodiment include top substrate thickness=0.2 mm, SRR layer thickness=ground plane thickness=0.0356 mm, bottom substrate thickness=0.76 mm, microstrip 202a width=1.4 mm, SRR height (all)=1.95 mm, via spacing=0.45 mm, unit cell size=3.8 mm, and SRR trace width=0.21 mm. Additionally, SRRs 206x have an interior opening width=1.98 mm and the capacitive loading ears have a height of 1 mm. SRRs 208x have an interior opening width of 1.53 mm and the capacitive loading ears have a height of 0.25 mm. SRRs 210x have an interior opening width of 0.78 mm. The gaps in all SRRs were set to 0.21 mm.

In operation, the split-ring resonators are excited by energy magnetically coupled through their respective microstrip lines, which are respectively connected to separate drive/sensing circuitry for each line. Strong E-fields are then generated across the gap between the loop terminals. When loaded by different MUT, the local E-field will be disturbed, resulting in frequency shift and attenuation that varies based on the MUT dielectric properties. By observing these differences, we can distinguish material properties.

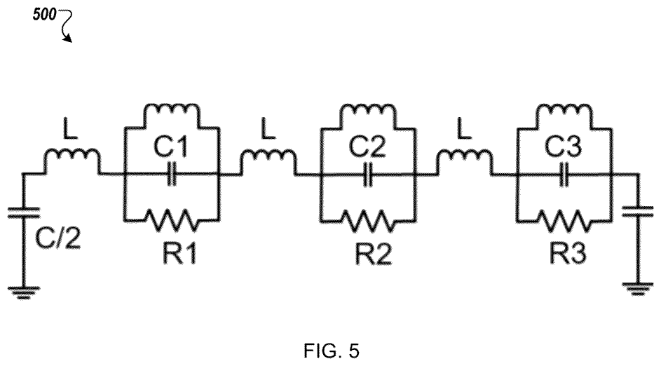

Each row of unit cells of the unloaded sensor board (without top substrate) can be modeled as the equivalent circuit 500 shown in FIG. 5. L and C model the effects of the transmission line, and each SRR can be modeled as a resonant tank using Ln, Cn, Rn (n=1, 2, 3). Extracted parameters using ADS: L=0.6 nH, C=8 pF, L1=0.097 nH, C1=3 pF, L2=0.045 nH, C2=4 pF, L3=0.025 nH, C3=3.65 pF, R1=285Ω, R1=255Ω, R1=165Ω.

To determine the sensing ability of the sensor array for skin diseases, we simulated and measured skin related tissue layers in CST Studio including both healthy and malignant tissue. By loading the MUTs across the sensing region, each SRR unit shows comparable performance, and resonance shift and attenuation due to the MUT loading and their dielectric properties are clearly seen.

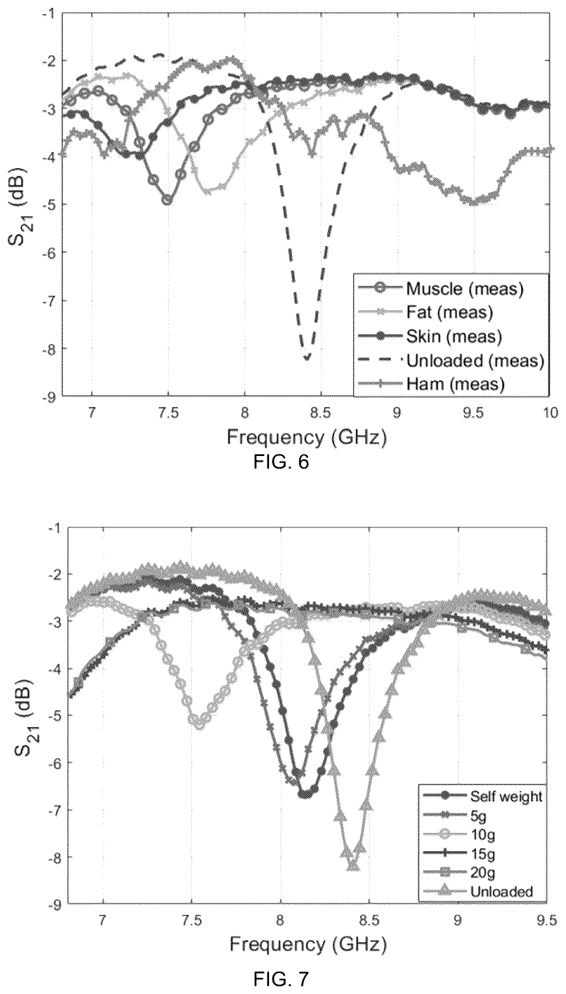

In FIG. 6, measured attenuation is plotted for different tissue samples and compared to unloaded attenuation, for a selected SRR as a function of frequency. As can be appreciated, each tissue sample provides a different characteristic attenuation profile that can be used in a classifier system to classify a type of tissue based on an observed profile.

Since contact pressure varies, it is critical to ensure a firm and repeatable contact between the MUT and SRR for accurate sensing. To characterize this dependence, we placed calibrated plastic weights on MUT to emulate different force levels while minimizing their influence on sensing results thanks to their low dielectric constants. The comparison for one resonator shows that ˜10 g of force is needed for repeatable readings. The resonance also significantly attenuates if larger forces are applied, due to deformation of the top substrate and metal layers under pressure. In FIG. 7, measured attenuation is plotted for a same tissue sample and different force levels, for a selected SRR as a function of frequency.

Hyperthermic ablation is a process to achieve cell death by elevating temperature around disease area, and it is demonstrated to be effective against malignancies. Since skin is rich in water content, microwave energy delivered from the SRRs can be coupled into tissue to generate heat. To characterize the hyperthermia process and associated dose, we used a measurement setup with chicken breast. We first measure the resonant frequency of a chosen SRR loaded with MUT, which will then be generated by a frequency synthesizer and amplified as the input power.

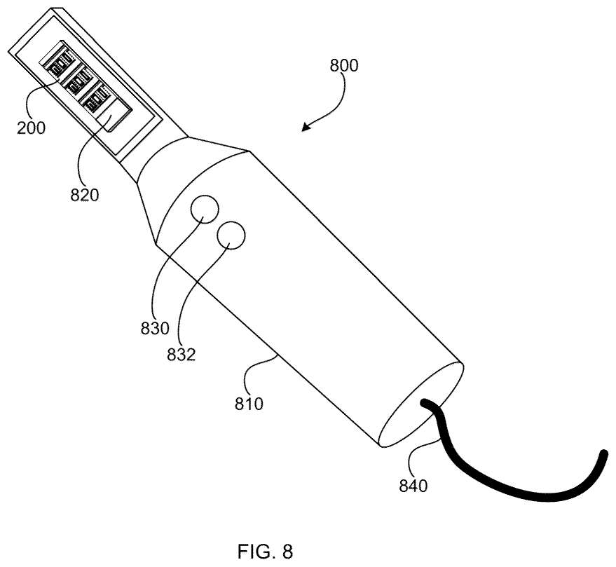

FIG. 8 illustrates one concept for a handheld applicator 800 useful in an embodiment. An SRR array 200 is mounted near one end of applicator 800, with a force regulation mechanism 820 that maintains a desired contact force with the MUT. Mechanism 800 may comprise, e.g., a spring-loaded translation mount, a linear active motor positioner with force feedback, or similarly operating components. The SRR array may also contain a thermocouple device (not shown) that provides temperature feedback during treatment.

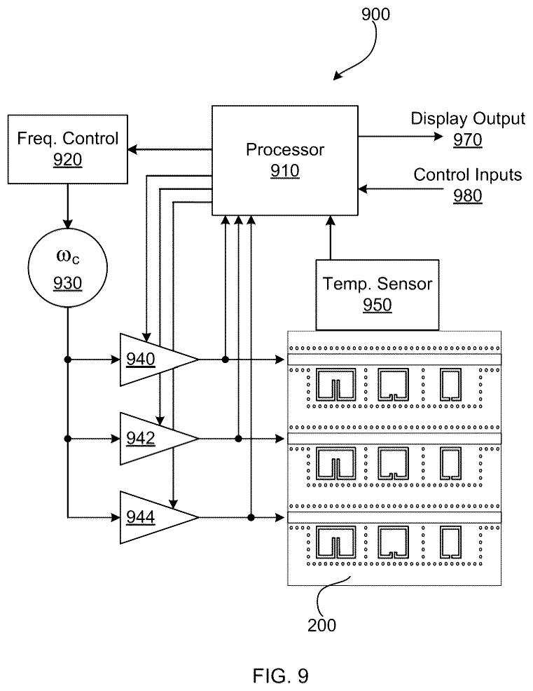

A housing 810 can, in some embodiments, hold some or all of the circuitry components (shown below in FIG. 9) that operate the SRR array. In such cases, cord 840 may merely provide power for the device. Alternately, some or all of the circuitry components (other than SRR array 200) may be located in a separate base unit (not shown). In this case, cord 840 may support, e.g., RF signaling for the SRR array, and potentially other control and measurement signals. Alternately, some or all control and measurement signals can be transmitted wirelessly to a base unit and/or display device (not shown). User controls 830, 832 disposed on applicator 800 can be used, e.g., to turn the device on and off, switch between a diagnostic mode and a treatment mode, and perform other control functions. Such user controls can alternately be provided in whole or in part on a base unit.

Alternate embodiments can substitute computer-controlled SRR array positioning for handheld operation, potentially in conjunction with an image recognition system or user guidance to place the SRR array in a region of interest.

FIG. 9 contains a block diagram for major electronic components of a system embodiment 900. A processor 910, which can be a programmed general purpose microprocessor, application-specific integrated circuit, field-programmable gate array, or similar circuit provides operational logic for system 900. Processor 910 control signals include a control signal to a frequency control circuit 920 and drive strength control signals to each of a set of RF amplifiers 940, 942, 944. Frequency control circuit 920 adjusts the frequency of a frequency generator that supplies a reference signal to each of the amplifiers (in alternate embodiments, each amplifier can have its own separate frequency generator and the frequencies are independently controllable).

While the frequency control determines which, if any, of the SRRs coupled to particular microstrip transmission line are active, the drive strength control signals determine a power setting for each amplifier. For instance, in a diagnostic mode, all three amplifiers can be driven at a low, mW-level power setting suitable for measuring a resonant shift and attenuation factor simultaneously for all rows. Feedback attenuation measurements are provided to the processor. In a treatment mode, however, amplifier 942 can be driven at a high-power setting (for instance 5 W) at or near a loaded resonant frequency of a selected SRR on the center row, while amplifiers 940 and 944 are either off or driven at a measurement power setting. It is also possible to drive multiple amplifiers on different rows simultaneously at a high-power setting for treatment of a larger area. If it is desired to treat a horizontally extended area, frequency control circuit 920 can be instructed to repeatedly shift between the loaded measured resonant frequencies of multiple SRRs along a selected treatment row or rows. A temperature sensor 950 can monitor local temperature during treatment to monitor elevation of a temperature of the treatment region on the skin.

During diagnostic mode, frequency control circuit 920 is instructed to slew frequency across a range of frequencies that allow capture of a characteristic attenuation profile for a given SRR on each of one or more SRR rows. Features extracted from a captured profile, including resonant peak shift, peak attenuation, shape and position of an attenuation trough, etc., are fed to a trained classifier to determine a probably tissue type (healthy skin, blemished but non-malignant skin, malignant lesion, etc.) for the tissue directly underlying the given SRR. This procedure can then be repeated for other SRRs along the rows by instructing control circuit 920 to slew frequency across a range of frequencies in which each SRR should exhibit a loaded resonant peak response.

In one embodiment, a display is generated that shows the classified tissue type for each SRR's current location, e.g., as a two-dimensional representation with colors or other markers to show classified tissue type at each location. Using visual feedback from such a display, an operator can make fine adjustments to applicator position so that a lesion is centered on the SRR array for treatment. During fine adjustment, the measurement procedure is recursed and the display is updated accordingly.

The disclosed applicator arrays are dual-mode, high sensitivity, and low-cost. The use of different SRR geometries along each line allows discrimination by unit cell, as each cell will be excited at a different frequency, and greatly reduces the size of the array and requirement for coupling connections. As both an imaging and treatment tool, this applicator encourages more accessible skin cancer management, especially in early stages. After exploring designs with two substrate materials and four different SRR geometries, a sensor board using FR-4 and rectangular loops is chosen for affordability, sensitivity and resonance characteristics, further enhanced by a via-fence structure. We further explore its sensing capability by loading different biological tissues on the sensor in both simulation and experiment, with results showing clear frequency shifts and attenuations to distinguish them from each other. Finally, we implement a measurement setup with live temperature monitoring to track thermal responses, and the results in both simulation and experiment demonstrate promising outcome for hyperthermia purpose.

Reference throughout the specification to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments. Further, it is intended that embodiments of the disclosed subject matter cover modifications and variations thereof.

It must be noted that, as used in the specification and the appended claims, the singular forms “a,” “an,” and “the” include plural referents unless the context expressly dictates otherwise. That is, unless expressly specified otherwise, as used herein the words “a,” “an,” “the,” and the like carry the meaning of “one or more.” Additionally, it is to be understood that terms such as “left,” “right,” “top,” “bottom,” “front,” “rear,” “side,” “height,” “length,” “width,” “upper,” “lower,” “interior,” “exterior,” “inner,” “outer,” and the like that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Furthermore, the terms “approximately,” “about,” “proximate,” “minor variation,” and similar terms generally refer to ranges that include the identified value within a margin of 20%, 10% or preferably 5% in certain embodiments, and any values therebetween.

All of the functionalities described in connection with one embodiment are intended to be applicable to the additional embodiments described below except where expressly stated or where the feature or function is incompatible with the additional embodiments. For example, where a given feature or function is expressly described in connection with one embodiment but not expressly mentioned in connection with an alternative embodiment, it should be understood that the inventors intend that that feature or function may be deployed, utilized or implemented in connection with the alternative embodiment unless the feature or function is incompatible with the alternative embodiment.

Moreover, the present disclosure is not limited to the specific circuit elements described herein, nor is the present disclosure limited to the specific sizing and classification of these elements.

While certain embodiments have been described, these embodiments have been presented by way of example only, and are not intended to limit the scope of the present disclosures. Indeed, the novel methods, apparatuses and systems described herein can be embodied in a variety of other forms; furthermore, various omissions, substitutions and changes in the form of the methods, apparatuses and systems described herein can be made without departing from the spirit of the present disclosures. The accompanying claims and their equivalents are intended to cover such forms or modifications as would fall within the scope and spirit of the present disclosures.

What is claimed is:

1. A dual-mode medical device comprising:
a resonator array comprising
a dielectric substrate having upper and lower surfaces,
a ground plane disposed on the lower surface,
a plurality of microstrip transmission lines disposed on and spaced across the upper surface, and
for each respective one of the plurality of microstrip transmission lines, a respective plurality of resonators disposed along the upper surface and adjacent that microstrip transmission line in a configuration supporting radio frequency (RF) energy coupling between each resonator of the respective plurality of resonators and the adjacent microstrip transmission line;
wherein, within each respective plurality of resonators, each resonator of the respective plurality has a different configuration than other resonators of the respective plurality, such that each resonator of the respective plurality has a different unloaded resonant frequency than other resonators of the respective plurality.

2. The dual-mode medical device of claim 1, further comprising, interposed between adjacent resonators of each respective plurality of resonators, a respective via fence comprising a spaced plurality of conductive vias formed in the dielectric substrate and in electrical contact with the ground plane.

3. The dual-mode medical device of claim 2, further comprising at least one respective additional via fence interposed between each given microstrip transmission line of the plurality of microstrip transmission lines and any resonator in the resonator array that is proximate to but not intended to couple to that given microstrip transmission line.

4. The dual-mode medical device of claim 1, wherein each of the resonators in the resonator array is a split-ring resonator (SRR).

5. The dual-mode medical device of claim 4, wherein at least some of the SRRs in the split-ring resonator array comprise a rectangular loop structure with a gap having capacitive loading ears.

6. The dual-mode medical device of claim 1, further comprising a top dielectric layer overlying the plurality of microstrip transmission lines and the plurality of resonators of each microstrip transmission line of the plurality of microstrip transmission lines.

7. The dual-mode medical device of claim 1, further comprising a controller circuit to selectively supply, to each of the plurality of microstrip transmission lines, a respective radio frequency (RF) signal.

8. The dual-mode medical device of claim 7, wherein the controller circuit is configured to, in a diagnostic mode where the resonator array is placed in contact with a patient's skin, perform a first resonator-selective procedure, the first resonator-selective procedure comprising:
sweeping a frequency of the respective RF signal supplied to a given one of the plurality of microstrip transmission lines, over a range of frequencies within which a loaded resonant frequency is expected to be found for a selected resonator that couples to the given one of the plurality of microstrip transmission lines;
detecting a current loaded resonant frequency for the selected resonator; and
classifying a tissue condition of the patient's skin at a current position of the selected resonator based at least in part on the current loaded resonant frequency.

9. The dual-mode medical device of claim 8, wherein the first resonator-selective procedure further comprises:
measuring an attenuation of the respective RF signal at the current loaded resonant frequency; and
classifying the tissue condition further based on the attenuation.

10. The dual-mode medical device of claim 8, wherein the controller circuit is further configured to, in the diagnostic mode, repeat the first resonator-selective procedure for each other combination of a given one of the plurality of microstrip transmission lines and resonator that couples to that given one of the plurality of microstrip transmission lines.

11. The dual-mode medical device of claim 10, wherein the controller circuit is further configured to, in the diagnostic mode, generate a two-dimensional graphical representation of classified tissue condition for each resonator in the resonator array and the current position of that resonator.

12. The dual-mode medical device of claim 11, wherein the controller circuit is further configured to, in the diagnostic mode:
recurse the first resonator-selective procedure for each resonator in the resonator array; and
update the two-dimensional graphical representation of classified tissue condition for the current position of each resonator.

13. The dual-mode medical device of claim 8, wherein the controller circuit is further configured to, in a treatment mode, perform a second resonator-selective procedure, the second resonator-selective procedure comprising:
selecting a treatment frequency at or near the current loaded resonant frequency of a selected one of the resonators of the resonator array; and
applying RF energy at a treatment power to the given one of the plurality of microstrip transmission lines that couples to the selected one of the resonators to perform a hyperthermic treatment of the patient's skin at the current position of the selected one of the resonators.

14. The dual-mode medical device of claim 13, further comprising a temperature sensing circuit to monitor the patient's skin temperature at or near the current position of the selected one of the resonators.

15. The dual-mode medical device of claim 13, wherein the controller circuit is further configured to, in the treatment mode, perform the second resonator-selective procedure for one or more other ones of the resonators of the resonator array.

16. The dual-mode medical device of claim 13, wherein the controller circuit is further configured to, concurrently with the treatment mode using the selected one of the resonators, perform the first resonator-selective procedure for one or more of the resonators of the resonator array.

17. The dual-mode medical device of claim 1, further comprising an applicator to hold the resonator array during positioning of the medical device against a patient's skin.

18. The dual-mode medical device of claim 17, wherein the applicator movably holds the resonator array, and adjusts a position of the resonator array relative to at least one other part of the applicator to attempt to maintain a constant contact pressure of the resonator array against a patient's skin.

19. The dual-mode medical device of claim 1, wherein the unloaded resonant frequencies of the resonators of the resonator array lie in a range between 5 GHz and 15 GHz.

20. The dual-mode medical device of claim 1, wherein the unloaded resonant frequencies of the resonators of the resonator array lie in a range between 1 GHz and 25 GHz.

21. A method of imaging and treating skin conditions, comprising:
providing a control for an operator of a dual-mode medical device to select between one of at least a diagnostic mode and a treatment mode for the dual-mode medical device;
in the diagnostic mode, while a two-dimensional array of resonators is in contact with a patient's skin, performing a first resonator-selective procedure for each given resonator in the two-dimensional array of resonators, wherein the first resonator-selective procedure comprises
sweeping a frequency of a respective RF signal supplied to a microstrip transmission line coupled to the given resonator, over a range of frequencies within which a loaded resonant frequency is expected to be found for the given resonator,
detecting a current loaded resonant frequency for the given resonator, and
classifying a tissue condition of the patient's skin at a current position of the given resonator based at least in part on the current loaded resonant frequency; and
in the treatment mode, while the two-dimensional array of resonators is in contact with the patient's skin, performing a second resonator-selective procedure for at least one resonator in the two-dimensional array of resonators, wherein the second resonator-selective procedure comprises
selecting a treatment frequency at or near the current loaded resonant frequency of a selected one of the resonators of the two-dimensional array of resonators, and
applying RF energy at a treatment power to the microstrip transmission line that couples to the selected one of the resonators to perform a hyperthermic treatment of the patient's skin at the current position of the selected one of the resonators.

22. The method of claim 21, wherein multiple resonators of the two-dimensional array of resonators couple to a same microstrip transmission line, the multiple resonators that couple to the same microstrip transmission line having mutually differing physical configurations that result in each of the multiple resonators having an unloaded resonance frequency range that is substantially non-overlapping with an unloaded resonance frequency range of each other resonator of the multiple resonators.

23. The method of claim 22, wherein the unloaded resonant frequency ranges of the multiple resonators each lie in a range between 5 GHz and 15 GHz.

24. The method of claim 21, further comprising, in the diagnostic mode, generating a two-dimensional graphical representation of classified tissue condition for each respective resonator in the two-dimensional array of resonators and the current position of the respective resonator.
